(19) United States
(12) Patent Application Publication
(10) Pub. No.: US 2015/0343189 A1
(43) Pub. Date: Dec. 3, 2015

(54) METHODS AND HEAD-MOUNTED DEVICES FOR PAIN RELIEF BY STIMULATING TREATMENT LINES ALONG THE SCALP

(71) Applicant: Igor Ostrovsky, Brooklyn, NY (US)

(72) Inventor: Igor Ostrovsky, Brooklyn, NY (US)

(21) Appl. No.: 14/294,069

(22) Filed: Jun. 2, 2014

(51) Int. Cl. A61M 37/00; A61B 18/04; A61N 1/04; A61M 5/00; A61H 39/08; A61B 18/06; A61M 19/00; A61N 2/00; A61H 23/00; A61N 2/02; A61N 5/06

(52) U.S. Cl. CPC A61M 37/00; A61N 2/02; A61B 18/042; A61N 1/0456; A61M 5/00; A61N 5/0616; A61B 18/06; A61M 19/00; A61N 2/002; A61H 23/00; A61H 39/08; A61B 2018/00321

(57) ABSTRACT

A method for relief of pain comprises applying skin stimulation along one or two treatment lines traversing vertically the scalp of the subject above the eyes thereof. Skin stimulation may be accomplished by one or several techniques including acupuncture, corona discharge, applying an electromagnetic field, massaging, vibration, subcutaneous injection of stimulating fluids, light, or heating. Suitable head-mounted devices supporting skin stimulation units are described to allow self-use by the subject itself or the method to be administered by a medical personnel.

### BACKGROUND OF THE INVENTION

The present invention relates generally to skin stimulating methods and devices for relieving pain. More particularly, the invention describes methods for pain relief in various parts of the body by stimulating skin over specifically defined lines of the subject's scalp as well as various head-mounted devices configured for such stimulation of the skin.

Noninvasive and minimally invasive methods and devices for treating various types of persistent pain are being developed all the time as the need for such methods and device is increasing over time with more patients seeking relief which is not provided by other medical therapies.

The International Association for the Study of Pain (IASP) states that pain is “an unpleasant sensory or emotional experience associated with actual or potential tissue damage, or described in terms of such damage.” This describes pain as a subjective experience; therefore there is no objective measurement for pain intensity.

The most commonly experienced form of pain may be defined as the effect of a stimulus on nociceptive nerve endings, which results in the transmission of impulses to the brain. This important somatic sensation and normal processing of painful stimulus, referred to as nociception or nociceptive pain, informs the organism of impending tissue damage. In nociceptive pain, signal is transmitted to the dorsal horn of the spinal cord, and then to the brain via ascending cortical pathways. Nociceptive signals within the brain are sent to the somatosensory cortex, prefrontal cortex where the sensory component of pain is processed in respect to its localization and significance, and to the limbic system, which is the neural substrate for the emotional component of pain.

Neuropathic pain and psychogenic pain, may develop without actual impending tissue damage. Most chronic pains are neuropathic in nature. Neuropathic pain typically occurs following injury to elements of the nervous system involved in nociception, such as peripheral nerve injury in phantom pain, causalgia, peripheral neuropathy or postherpetic neuralgia. The neuropathic or psychogenic pain pathways are not associated with immediate action to prevent injury. The phantom pain experienced following amputation of a limb, for example, is neuropathic—there is no danger of injury to the missing limb. Psychogenic pain is a chronic condition without definite organic pathology.

Being a subjective phenomenon, pain is experienced differently by different subjects even in a similar circumstances. Patient's interpretation of painful sensation in the context of previous life experience, its significance for health, well-being and survival may lead to exacerbation of painful experience or on the contrary, to diminished reaction to pain. Individual variations of the levels of excitatory and inhibitory neurotransmitters in the spinal cord and the brain may also explain the differences in the tolerance for pain among individuals.

Diagnosis and evaluation of pain is based on complex interpretation of patient's subjective complaints, the underlying pathology and various other factors. Treatment of pain depends on understanding of the underlying organic basis of the pain, and is particularly difficult in patients with chronic pain syndromes, because chronic pain may lack an apparent underlying anatomical cause.

Common painful complaints include low back and neck pain, headache, facial pain, and pain in the extremities. Chronic pain which lacks pathological basis is sometimes psychogenic, and may be due to anxiety, depression, hysteria, hypochondria, or simply malingering. Chronic pain may also be neuropathic, as in causalgia, or peripheral neuropathy. Neuropathic pains are treated with analgesics, opioids, antidepressants and injections of local anesthetics.

Pharmacologic therapy is the principal form of treatment for pain today, including widespread use of analgesics, narcotics, corticosteroids, antidepressants, topical anesthetics and local anesthetic injections. In an acute setting, such as after surgery, narcotics and anti-inflammatory drugs are used. Other drug therapies include tricyclic antidepressants which help to provide analgesia by activation of the descending pathways. In some cases, local or systemic administration of anesthetic agents are used. For patients with intractable pain, radiofrequency nerve ablation, intrathecal opiates, spinal cord stimulators, transcutaneous nerve stimulators, implantable electrical stimulators, deep brain stimulators and high intensity magnetic field stimulators may be used.

Debilitating chronic pain affects tens of millions of people annually. According to National Center for Technology and Information study published in 2010, as many as 100 million Americans suffer from chronic pain yearly, and the total financial cost of pain to society, which combines the health care cost estimates and the three productivity estimates, ranges from $560 to $635 billion. The annual cost of pain was greater than the annual costs in 2010 dollars of heart disease ($309 billion), cancer ($243 billion), and diabetes ($188 billion) and nearly 30 percent higher than the combined cost of cancer and diabetes. An IOM (Institute of Medicine) committee commissioned by Congress concluded that pain is not optimally managed in the US. Pain also contributes to national rates of mortality. According to CDC, in 2008 nearly 15,000 deaths were attributed to prescription pain killers, which exceeds deaths involving heroin and cocaine combined.

Given the magnitude of the economic and health costs of pain there is an urgent need for safe and effective devices and methods for treating chronic pain that may reduce the need for habit-forming pain-relieving drugs.

Acupuncture is a well-known treatment method used for pain relief and for other medical conditions. Stimulation of acupuncture points produces release of certain chemicals (e.g., endorphins, neurotransmitters, hormones). These chemicals may enhance the body's ability to heal itself. Modern research suggests various mechanisms of acupuncture effects on pain and body functions. These mechanisms include activation of endogenous opioid system, complex reactions involving all levels of peripheral and central nervous system from primary afferents to multiple brain areas. In brief, acupuncture effects on the nervous, endocrine and immune systems lead to change in perception and processing of painful stimuli.

New points and areas of skin stimulation are being sought to further advance the efficacy of pain relief. One desirable feature of a skin stimulation area is to make it universal so as to provide pain relief in various parts of the body by stimulating the same area of the skin. Finding such universal area may go a long way in providing pain relief without the need to see a specialist or with a minimum training required to master such skin stimulation.

Despite the advancements of acupuncture over thousands of years, it still has not reached the level of being available to the subject for self-use. Deep knowledge of appropriate stimulation points mastered by practitioners of this art are not available to the general public. In general medical community there is also lack of knowledge in respect to acupuncture points and its use for treatment of pain and other medical conditions. The need exists therefore for a device suitably designed for applying pain-relieving methods of skin stimulation by the subject himself, and by medical professionals with minimal training in the field of acupuncture.

### SUMMARY OF THE INVENTION

Accordingly, it is an object of the present invention to overcome these and other drawbacks of the prior art by providing novel methods of alleviating pain applicable to a broad range of painful and other medical conditions.

It is another object of the present invention to provide novel methods of reducing pain which are easy to teach to a novice in this field.

It is a further object of the present invention to provide novel devices for reducing pain configured for use in a doctor's office as well as by self-use.

It is yet a further object of the present invention to provide novel methods and devices for relieving pain that can be activated on a periodic basis making them suitable for treating chronic pains of various types.

The methods of the invention describe an unexpected effect of stimulating skin over a newly defined area of skin along the forehead of a subject's scalp. More specifically, the novel methods of relieving pain according to the invention involve a step of stimulating skin on a scalp of a subject along at least a 2 cm long or longer portion of a treatment line along the scalp. The treatment line is defined as at least a 6 cm long line located along a vertical line drawn along the scalp through at least one of a left pupil or a right pupil of the subject and upwards along the scalp. The treatment line is defined as being between a first point on this vertical line located on the forehead at 2 cm below an actual or estimated hairline and a second point on the vertical line located at 4 cm above the actual or estimated hairline of the subject.

Two treatment lines may therefore be defined for a particular subject—one on the left side and one on the right side. Skin stimulation along at least one of them may be used for pain relief. In embodiments, pains encountered on the right side of the body may be treated by skin stimulation along the treatment line of the left side of the scalp—and vice versa. Centrally located pain may be alleviated using either one or both treatment lines.

Devices for self-administering skin stimulation for pain relief according to the present invention are also described. Such devices include a head-mounted base and one or two skin stimulation units adjustably attached to the base and configured to facilitate applying skin stimulation to the treatment lines described by the methods of the invention.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is a front view of the head of a subject with outlined treatment lines according to the present invention;

FIG. 2 is a ¾ view of a head of another subject with an estimated hairline showing how to define the treatment lines along a scalp thereof;

FIGS. 3a, 3b, and 3c show variations of electrodes usable with skin stimulation devices of the present invention;

FIG. 4 schematically shows a first embodiment of the head-mounted device for stimulating skin of a subject for the purposes of relieving pain;

FIG. 5 shows the first embodiment of this device in use on a head of the subject;

FIG. 6 schematically shows a second embodiment of the device of the invention; and

FIG. 7 shows the second device in use over the head of the subject.

### DETAILED DESCRIPTION OF THE PREFERRED EMBODIMENT OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

A broad range of acute and chronic pains and certain other medical conditions may be relieved using the methods of the present invention. Some examples of pains as well as related undesirable sensations and medical conditions that may be treated using the present invention include the following:
a. acute and chronic pains in the neck, low back and limbs
b. neuralgic pains
c. neuropathic pains
d. migraine and tension headaches
e. painful menstrual cramps
f. preterm labor or threatened abortions
g. muscle cramps in the limbs and elsewhere
h. morning sickness in pregnant women
i. fatigue
j. insomnia
k. anxiety
I. irritability
m. vertigo

The list above is not limited to just these specifically mentioned types of pains. Other acute and chronic painful conditions may also be alleviated using the described methods as the present invention is not limited in this regard.

### Definition of Treatment Lines

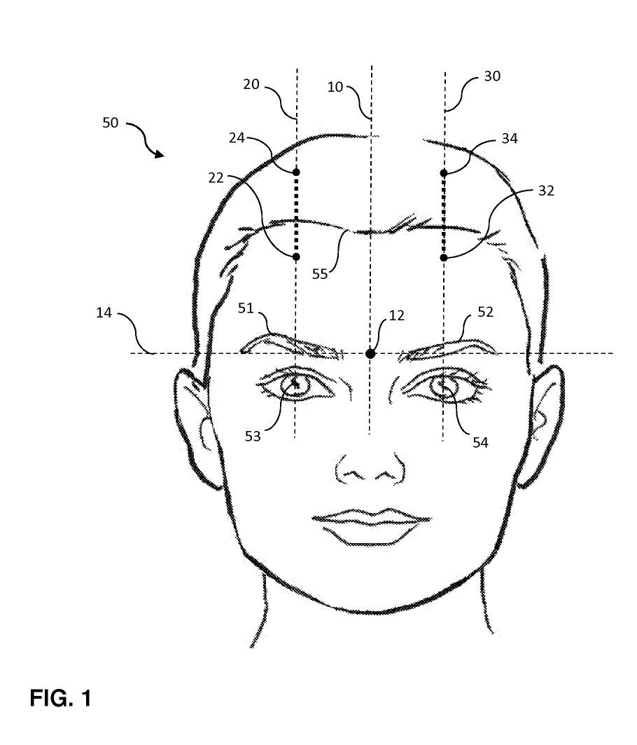

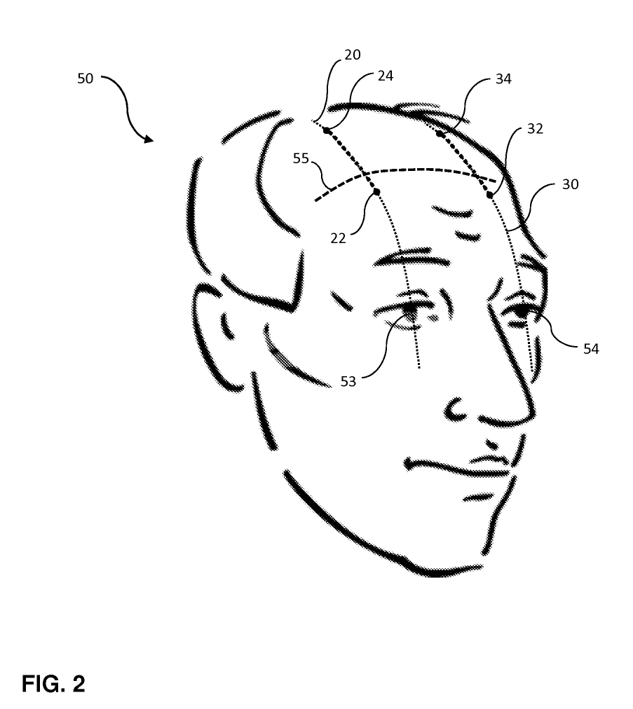

FIGS. 1 and 2 show a front view and a ¾ view of a typical head 50 of a subject for the purposes of illustrating how to define the novel treatment lines unexpectedly discovered by the present inventor. One such line may be located on the left side of the head 50 and another treatment line may be located symmetrically on the right side of the head 50.

To define the location of the treatment lines takes a few simple steps. First, a left pupil 54 and the right pupil 53 may be identified when the subject is asked to look straight forward in front of him.

After that one vertical line 20 is hypothetically drawn through the right pupil 53 and another vertical line 30 is hypothetically drawn through the left pupil 54. Both vertical lines 20 and 30 are drawn along the skin upwards from the corresponding pupil along the forehead and the rest of the scalp of the head 50.

Following establishment of at least one of the lines 20 or 30, the hairline 55 of the subject may be examined. In some subjects with full head of hair, such hairline 55 may be very apparent visually—as seen in FIG. 1. In other subjects, however, such as those with partial or complete baldness, there may be no visible hairline 55—see FIG. 2. In this case, a projected location of a normal hairline may be established as seen using a dashed line 55 in the example shown in FIG. 2.

The points of intersection of the hairline 55 and the vertical lines 20 and 30 may then be established. According to the methods of the present invention, a treatment line may be established to be located along the vertical line through a pupil of the subject as follows: it starts at a first point about 2 cm below the hair line and ends at a second point about 4 cm above the hairline. The term “about” is used throughout this description to describe a 10% variation of the respective measure. As seen in FIGS. 1 and 2, the left treatment line may extend from the first point 32 to a second point 34 along the vertical line 30, while the right treatment line may extend from a first point 22 to a second point 24 along the vertical line 20. Bot treatment lines are therefore about 6 cm long.

According to the methods of the present invention, to reach a meaningful pain relieving effect, it is not necessary to stimulate skin along the entire length of the treatment line. Rather, skin stimulation along at least a 2 cm portion of the treatment line may provide acceptable results. This 2 cm long portion may be selected anywhere along the 6 cm length of the treatment line.

While best results may be obtained with skin stimulation along these treatment lines, small deviations to the left side or the right side of the treatment line may be acceptable. In embodiments, such deviations may not exceed 5 mm on each side without losing at least some portion of the treatment effect.

In addition to skin stimulation along at least one or both of the treatment lines described above, a new point of skin stimulation 12 (see FIG. 1) causing relief of the same medical conditions was also unexpectedly discovered to be located at the intersection of the central vertical line 10 and the horizontal line 14 between the subject's eyebrows 51 and 52. This additional point of skin stimulation 12 may be utilized together with skin stimulation along the treatment lines described above, or a standalone method of obtaining pain relief.

As the methods of the invention may be applicable to a broad range of pain syndromes, it may be advantageous to separate the pain located anywhere along the central vertical line hypothetically drawn through a subject's body from those pains located on the right side or on the left side of the body. In embodiments, those pains located on the right side of the subject may be better treated by skin stimulation along the treatment line as described above located on the left side of the scalp of the subject. Those pains located on the left side of the subject may be best treated by skin stimulation along the treatment line located on the right side of the subject's scalp. Finally, centrally located pains may be treated by stimulating at least one or both of the right or left treatment lines.

Although relief may be apparent as a result of a single treatment as described above, repeated treatments may be applied to those pains that are more resistant to a single treatment. In embodiments, skin stimulation may be conducted repeatedly over a course of pain relief treatment, such course may include a number of skin stimulation treatments ranging from 2 to 10 applied over a duration of treatment ranging from 2 to 20 days at a rate of 1 treatment every other day to 2 treatments daily.

### Skin Stimulation Techniques

Various types of skin stimulation techniques may be applied along the treatment lines described above for providing durable pain relief for the subject. Importantly, these techniques may be designed to stimulate the skin but not cause any lasting skin damage to avoid any safety concerns for the subject. In embodiments, the following skin stimulation techniques may be applied:

a. Applying Corona Discharge

Stimulation of the skin along the treatment line with corona discharge may be accomplished between a surface of a glass electrode (described in more detail below) and the skin of the subject, which may be further supplemented by the electromagnetic field generated inside the electrode. Typically, such electrodes may be either vacuumed to remove air or filled with an inert gas such as Argon or Neon to replace air. Periodically variable electrical current using for example bell-shaped pulsations may be passed through the electrodes of the glass tube. Such electrical current may be passed at a frequency of about 85 kHz to about 135 kHz using a voltage from about 7 kV to about 30 kV, and a safely low current of about 0.02 mA (ranging from about 0.018 mA to about 0.022 mA) for example. The alternating electric current may then be modulated with a pulse length of about 100 microseconds (ranging from about 90 microseconds to about 110 microseconds) using a pulse frequency of from about 50 Hz to about 100 Hz. This high voltage/low current electrical signal may be used to generate corona discharge in the air separating the surface of the glass electrode from the skin of the subject. The treatment effect of the methods of the invention may be attributed to the high frequency current likely propagating locally through the skin of the subject as well as to the direct action of the corona discharge on the skin receptors and the superficial tissues.

b. Applying Electromagnetic Field

Electromagnetic stimulation may be applied for a duration from about 1 minute to about 7 minutes in the range of about 25-120 milligauss using general principles described in the U.S. Pat. No. 5,085,626 incorporated herein by reference in its entirety.

c. Applying Electrical Current

This skin stimulation technique may be accomplished by positioning two electrodes at the beginning and at the end of the treatment line. Electrodes configured for not penetrating the skin as well as acupuncture needles may be used for such purpose. Once the electrodes are placed, electrical current may be applied therebetween. In one example of not penetrating skin electrodes, a Transcutaneous Electrical Nerve Stimulator (TENS) may be used to operate at a frequency range from about 2 Hz to about 150 Hz, current intensity from above 0 mA to about 80 mA, and a pulse width from about 5 milliseconds to about 250 milliseconds. For skin penetrating needle stimulation, the frequency of the electrical current may be selected to be from about 2 Hz to about 9 KHz, current intensity from about 2 mA to about 15 mA, and pulse width under 1 millisecond.

d. Acupuncture, Electroacupuncture or Acupressure

Skin stimulation for a period of about 10 min to about 30 min using acupuncture needles may be accomplished by inserting the needles underneath the skin at the beginning and at the end of the treatment zone. The treatment may be applied daily or once every other day. Alternatively, each needle may be tunneled superficially underneath the skin along the direction of the treatment line.

For the purposes of electroacupuncture, the needles or other non-skin-penetrating electrodes may be placed at both ends of the treatment line and an electrical current may be passed therethrough as described above.

Acupressure may be applied using sufficiently sharp objects pressed against the skin but not penetrating through the skin. Positions for such objects may be selected to be similar to that of acupuncture needles. In embodiments, rolling instruments with protruding acupressure spikes may be repeatedly moved back and forth along the treatment line. In other embodiments, percussion instruments may be used for this purpose.

e. Injection of a Stimulating Fluid Along the Treatment Line

An inert or mildly irritating biocompatible sterile fluid may be injected along the treatment line for the purposes of skin stimulation. Examples of such fluid include local anesthetic or a 5% dextrose solution. Lidocaine may be used as such local anesthetic. As compared to other skin stimulation techniques, this method has an advantage of extended time of action, spanning well beyond the initial time of fluid injection until the fluid is absorbed by the skin and underlying tissues.

f. Infrared Light or Laser Stimulation

Applying about 1 to about 5 minutes of light stimulation to the skin along the treatment line may be accomplished for example by using a Red laser (emanating light at a frequency of about 635 nM) or a blue laser (emanating light at a frequency of about 450 nM). In all cases, the output power may be selected to be below 100 mW, such as for example from about 2 mW to about 10 mW to assure subject's safety. Infrared light may also be used, such as for example emanating from an infrared-generating mercury lamp of a plurality of infrared LEDs configured to illuminate the treatment line on the subject.

g. Massaging or Vibration of Skin Along the Treatment Line

Various known in the art skin massagers or vibrators may be used for the purposes of this invention as long as they provide sufficient and effective skin stimulation. Examples of such devices include electrically-powered rotating weights, such weight may be configured to rotate about an axis line drawn away from their center of gravity. Pneumatically-activated massagers such as those releasing streams of air on a periodic basis aimed at the skin along the treatment line may also be used for the method of the present invention. In embodiments, other devices such as those containing a plurality of fingers configured to stay in contact with the skin and move it back and forth on a periodic basis may be used to cause skin stimulation.

h. Applying Heat or Cold Along the Treatment Line

Applying heat or cold to the skin once or on a repeatable periodic basis throughout the treatment session may be used as an effective non-invasive technique of skin stimulation. For safety reasons, the temperature of the skin should remain from about 30 degrees C. to about 45 degrees C. Various contacting and non-contacting skin heating and cooling techniques may be used for the present invention, such as suitably shaped electrical heaters, Peltier coolers, etc. To be effective, the temperature of the skin along the treatment line need to be changed by at least about two degrees C. from its assumed normal temperature of about 37 degrees C.

In further embodiments of the invention, suitable skin heating may be applied by moxibustion of moxa made from dried mugwort. To apply indirect moxibustion, one may hold a cigar made of mugwort near the acupuncture point to heat the skin, or hold such cigar on an acupuncture needle inserted in the skin to heat the needle. Direct moxibustion may not be suitable for the purposes of the invention if hair is present near the treatment line.

i. Applying Skin Compression, Pinching or Suction Along the Treatment Line

In embodiments, a wide variety of mechanical, electrically-powered and pneumatically powered instruments may be used to apply periodic or constant skin compression, pinching or suction along the treatment line. Care should be taken however to not cause lasting skin damage in the process.

### First Embodiment of the Invention

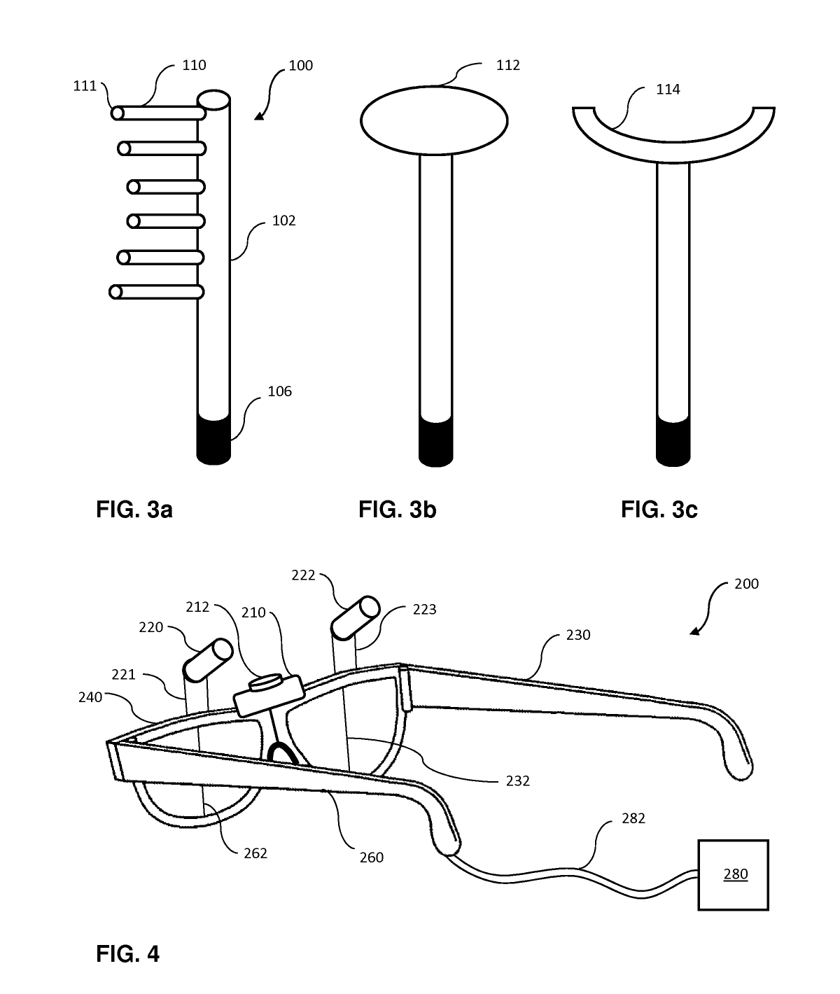

The first embodiment of the invention is generally described below and illustrated in FIGS. 3-5. While the description discusses Corona discharge electrodes and systems, this is used only as an example of utilizing one skin stimulation technique. Other skin stimulation techniques are also contemplated as part of the technology described below with suitable changes to the skin stimulation unit.

A corona is a general process by which a current develops from an electrode with a high potential in a neutral fluid (such as air), by ionizing the fluid to create a plasma around the electrode. The ions generated will pass charge to adjacent areas of lower potential. Corona discharge as a skin stimulation technique is generally known in the art. Typically, glass electrodes may be used for this purpose.

The term “corona discharge” is used broadly in this description to encompass electromagnetic and/or spark stimulation applied to nearby skin by a glass electrode energized by the control unit using so-called high-frequency currents causing damped electrical oscillations. Such high-frequency currents are also referred to as d′Arsonval currents, Oudin currents, and Tesla currents.

The shape and construction of the glass electrodes depends on where they will be used. FIGS. 3a-3c show examples of electrodes 100 suitable for the purposes of the invention. Generally speaking, an electrode 100 is made from a glass tube, such as for example having 1-2 mm thick glass wall. The glass tubes may be shaped at high temperature, by blowing and using templates. Electrode 100 has a structural portion 102 and metal electrode 106 which may be assembled at the bottom of the glass piece. Electrode 100 may be configured to be removably inserted into a mating well of the holder, which is in turn connected to a generator of electrical signals.

During manufacturing of the electrode, a small glass tube may be temporarily left at the bottom of the electrode 100 to communicate to the inside volume thereof. A vacuum may then be applied through the small tube to remove air and then that small tube may be used to fill the electrode with an inert gas, such as argon or neon. After filling, the tube may be closed off and removed by melting the glass.

When the electrode 100 is energized, the inert gas is ionized, causing a relatively small voltage drop across the ionized gas and a relatively larger voltage drop caused by passing through the thickness of the glass of the electrode. Glass thickness may be optimized to provide adequate strength and voltage drop. The skin stimulation effect of the method may be attributed to the action of the corona discharge (sparks) at skin receptors and at superficial tissues, and the high frequency current running in the skin. These phenomena may produce local vasodilation as well as other local stimulation effects.

The distal portion of electrodes 100 may be shaped to match the profile of the skin to be stimulated. FIG. 3a shows an example of the electrode 100 with the distal section containing a series of protrusions 110. To match the expected curvature of a human scalp, protrusions 110 may contain ends 111 located along an arch line, for example with a radius ranging from about 3 inches to about 15 inches. An alternate configuration is shown in FIG. 3b in which the distal portion of the electrode 100 contains a flat disk 112. Finally, FIG. 3c shows an arch-shaped distal portion 114 configured to be placed along the expected arch-shaped treatment line along the scalp of the subject. The radius of the arch may also be in the range from about 3 inches to about 15 inches.

As described above, the two treatment lines may be drawn through the pupils of the subject. As can be easily appreciated by those skilled in the art, human subjects have a distance between pupils which is variable to some extent and so the device 200 of the invention may be configured to retain one or two skin stimulation units 100 at the respected treatment lines with the provision to adjust distance therebetween—so as to allow customization of use for individual subjects.

In embodiments, the head-mounted base may be made using a frame of the glasses 200, such frame comprising two sides 230 and 260 to retain glasses 200 on the subject and a nose bridge portion 240 therebetween. The nose bridge portion 240 may support one or two holders 220 and 222 extending upwards from the glasses 200 and adapted to adjustably and removably retain skin stimulation units such as electrodes 100 therein. In embodiments, holders 220 and 222 may be attached to the glasses 200 via respective articulating joints 221 and 223 so as positions of electrodes 100 may be manually shifted.

In embodiments, the nose bridge portion 240 may be made of fixed length or with adjustable length. Fixed-length design may be configured to provide distance between holders 220 and 222 of about 60 mm, which is an average distance between the pupils of a human subject.

In other embodiments, to provide an ability to adjust the distance between electrodes 100 when retained in the holders 220 and 222, the nose bridge portion 240 may be equipped with a mechanism 210 operable by a rotating knob 212. Turning the knob may cause the left portion of the glasses 200 to move away from the right portion thereof. One example of the mechanism suitable for this purpose is described in the US 2002/0163486 incorporated herein in its entirety by reference.

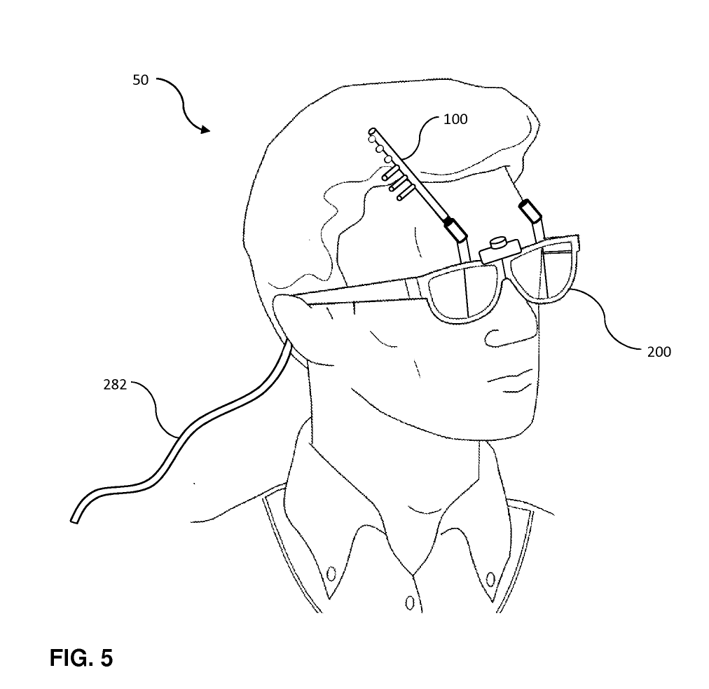

The glasses 200 may include vertical lines 262 and 232 drawn along the vertical planes through the centers of the respective holders 220 and 222. The lines 262 and 232 may be used to adjust the distance between the holders 220 and 222. When the glasses 200 are placed on the subject (see FIG. 5), the button 212 is turned until vertical lines 262 and 232 are positioned to be aligned to the subject's pupils. Such adjustment may be made by the user of the device, including self-adjustment by the subject when looking at the mirror.

The electrically-powered control unit 280 may be electrically coupled with the holders 220 and 222 by using a flexible cable 282 connecting the unit 280 to the glasses 200 of the invention. The length of the cable 282 may be selected to be sufficient to allow placement of the control unit 280 nearby the subject. The cable 282 may be made with provisions to prevent kinking or pulling on the glasses 200 when the device is in use, for example by providing at least a portion of the cable 282 to be coiled.

The control unit 280 may be configured to produce a high-frequency adjustable sinusoidal voltage waveform having a maximum amplitude ranging from about 7,000 V to 30,000 V, a frequency of about 50 kHz to 120 kHz, and a maximum output current of less than 0.1 mA. The electrical signal may be modulated to pulse length of about 100 microseconds at a suitable frequency ranging from about 50 Hz to 150 Hz.

In use, the glasses 200 may be positioned on a head of the subject 50 as seen in FIG. 5. The distance between the electrodes 100 may be adjusted using the mechanism 210 and the knob 212. Electrodes 100 may be positioned inside the holders 220 and 222 and their position may be adjusted to place the distal portion thereof along the projected treatment lines as described above. The control unit 280 connected to the glasses 200 via a cable 282 may then be activated to deliver appropriate skin stimulation according to the methods of the invention described above in greater detail.

### Second Embodiment of the Invention

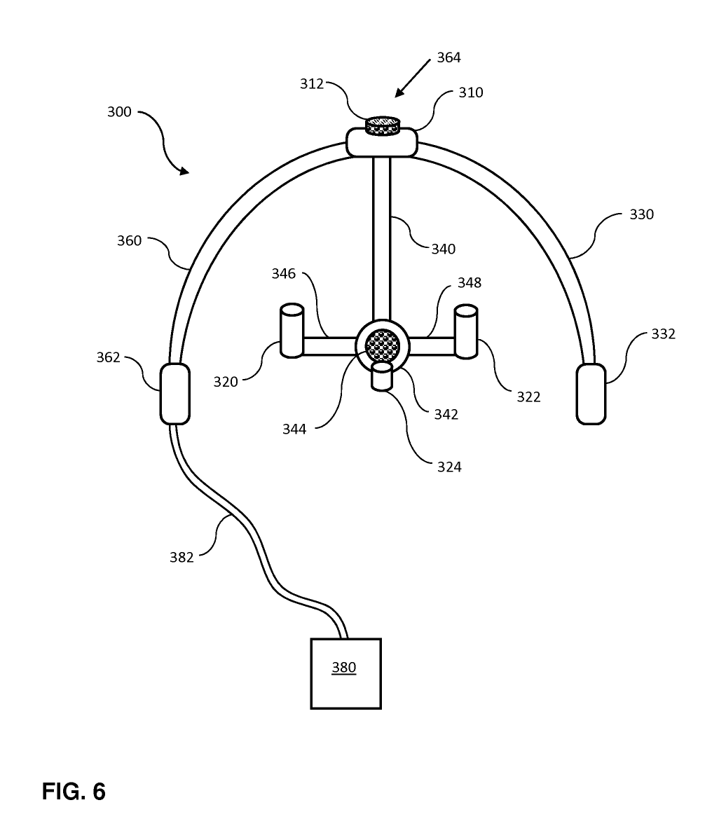

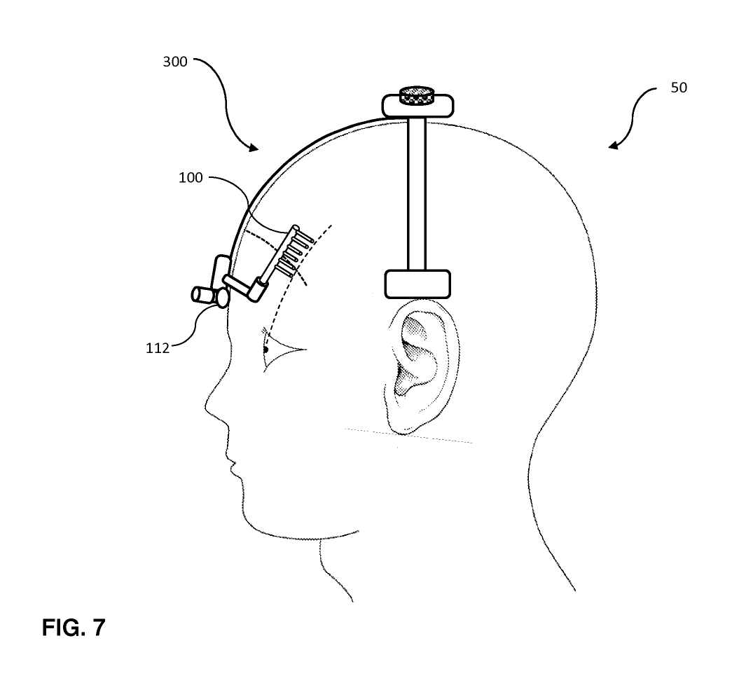

An alternative configuration of the head-mounted base of the invention is now described with references to FIGS. 6 and 7. Shown in FIG. 7 is an embodiment of the device comprising a head arch 300 configured to include a first portion 360 extending from a first ear terminal 362 to the midpoint 364 and the second portion 330 extending from a second ear terminal 332 towards the same arch midpoint 364. In some embodiments the head arch may have a fixed length and shape (not shown), while in other embodiments, the midpoint 364 may contain a length-adjustment mechanism 310, operable for example by rotating a knob 312 to tighten or loosen the head arch 300 when positioned on the head of the subject 50, see FIG. 7. The mechanism 310 may be selected to be similar to length-adjusting mechanisms of common headphones known in the art to support speakers about the ears of the subject. The arch 300 may be made from resilient materials such as polymers so as to snugly surround the head of the subject when in use.

A central arch 340 may also be attached to the midpoint 364 and configured to extend along the midline of the scalp towards the nose bridge of the subject. At the bottom end of the central arch 340 there is positioned an assembly configured to retain skin stimulation units along the treatment lines of the subject as described above. One example of such assembly is a first side bar 346 and a second side bar 348 extending outwardly towards the eyebrows of the subject from the bottom end of the central arch 340. Holders for skin stimulation units 320 and 322 may be positioned at the ends of the respective side bars 346 and 348. In some embodiments, the distance between the holders 320 and 322 may be fixed and selected to be about 60 mm. in other embodiments, the distance between the holders 320 and 322 may be adjustable via the mechanism 342 operable by turning a knob 344. The mechanism 342 may be made using a rake and pinion principles so as to move both side bars 346 and 348 symmetrically by the same distance. This may be done to retain the holders 320 and 322 symmetrically and at the same distance from the central arch 340.

The central arch 340 may further include a third holder 324 located along the central arch 340 and configured to retain a third skin stimulation unit to stimulate the point on the skin centrally located between the subject's eyebrows. One, two, or all three holders 320, 322, or 324 may be attached to the device 300 via respective articulating joints (not shown) so as to allow adjusting their position with regard to the location of the treatment lines along the scalp of the subject.

An optional back arch may also be used to extend from the midpoint 364 towards the back of the head of the subject (not shown). The back arch may be used to increase the stability of the device 300 when placed on the subject's head.

An electrically-powered control unit 380 of the design similar to that described for the first embodiment of the invention may be electrically coupled to the device 300 using a flexible cable 382.

In use, the arch 300 may be first positioned over the head of the subject 50, see FIG. 7. One or two electrodes 100 may be positioned in respective holders 320 and 322 and adjusted to touch the skin of the subject 50 along the desired skin treatment lines. A central skin stimulation unit 112 may also be positioned using the holder 324. The control unit 380 may then be activated to deliver the treatment according to the method described above in greater detail.

The herein described subject matter sometimes illustrates different components or elements contained within, or connected with, different other components or elements. It is to be understood that such depicted architectures are merely examples, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

Although the invention herein has been described with respect to particular embodiments, it is understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A method for relieving pain comprising a step of stimulating skin on a scalp of a subject along at least a 2 cm long portion of at least a 6 cm long treatment line, said treatment line located along a vertical line drawn through at least one of a left pupil or a right pupil of the subject and upwards along the scalp thereof, said treatment line located between a first point on said vertical line at 2 cm below an actual or estimated hairline and a second point on said vertical line at 4 cm above said actual or estimated hairline.

2. The method as in claim 1, further comprising a step of selecting a left side of the scalp for skin stimulation if said pain is located anywhere on a right side of said subject; or selecting a right side of the scalp for skin stimulation if said pain is located anywhere on a left side of said subject; or selecting both the left side of the scalp and the right side of the scalp for skin stimulation if said pain is located at a central location of said subject.

3. The method as in claim 1, wherein said skin stimulation is conducted repeatedly over a course of treatment, said course comprising a number of skin stimulation treatments ranging from 2 to 10 applied over a duration of treatment ranging from 2 to 20 days at a rate of 1 treatment every other day to 2 treatments daily.

4. The method as in claim 1, wherein said skin stimulation is applied along the treatment zone using one or a combination of skin stimulation techniques selected from a group consisting of:
applying corona discharge
applying electromagnetic field
applying electrical current
acupuncture, electroacupuncture or acupressure
injection of a stimulating fluid along the treatment line
infrared light or laser stimulation
massaging or vibration along the treatment line
applying heat or cold along the treatment line, and
applying skin compression, pinching or suction along the treatment line.

5. The method as in claim 4, wherein said stimulating fluid is a local anesthetic or a dextrose solution.

6. The method as in claim 4, wherein said skin stimulation is applied continuously for a period of time ranging from 1 minute to 7 minutes for electromagnetic stimulation, from 10 minutes to 30 minutes for acupuncture, electroacupuncture or acupressure, from 1 minute to 5 minutes for infrared light or laser stimulation.

7. The method as in claim 4, wherein said electric stimulation comprises insertion of electrode needles along said treatment line and applying electrical current therebetween.

8. The method as in claim 4, wherein said applying heat or cold is done by changing the skin temperature along said treatment line by at least 2 degrees above or below 37 degrees Celsius.

9. The method as in claim 8, wherein said applying heat is done using moxibustion of moxa made from dried mugwort.

10. The method as in claim 1 further comprising applying skin stimulation to a point on the subject's scalp centrally located between eyebrows thereof.

11. A head-mounted device for stimulating skin on a scalp of a subject, said device comprising:
a head-mounted base, and
a first skin stimulation unit attached to said head-mounted base such that at least one skin stimulating element is positioned along a skin stimulation treatment line, said treatment line is located along a vertical line drawn through at least one of a left pupil or a right pupil of the subject and upwards along the scalp thereof, said treatment line located between a first point on said vertical line at 2 cm below an actual or estimated hairline and a second point on said vertical line at 4 cm above said actual or estimated hairline of the subject.

12. The device as in claim 11 further comprising a second skin stimulation unit attached to said head-mounted base at a location symmetrical to said first skin stimulation unit relative to a vertical midline along the head of the subject, wherein said device is configured for skin stimulation along two parallel treatment lines above both pupils of the subject.

13. The device as in claim 12, wherein said first skin stimulation unit is located about 60 mm apart from said second stimulation unit.

14. The device as in claim 11, wherein said skin stimulation unit comprises a plurality of skin stimulating elements positioned along said treatment line.

15. The device as in claim 11, wherein said adjustable head-mounted base is a pair of glasses configured to provide support over the ears and a nose bridge of the subject, said first skin stimulation unit extending upwards from said pair of glasses along said treatment line.

16. The device as in claim 11, wherein said head-mounted base comprises a resilient head arch configured to be positioned over a head of the subject extending from one ear to the other ear, said head-mounted base further comprising a central arch extending from a midpoint of said head arch towards a nose bridge of the subject, said first skin stimulation unit attached to said central arch and extending to the right side or to the left side thereof along said treatment line.

17. The device as in claim 11, wherein said skin stimulation element comprises a sealed chamber filled with inert gas and at least one electrode, said device further including an electrically-powered control unit connected to said electrode with a flexible cable, said control unit is configured to generate an electrical signal to cause said electrode to generate at least one of a corona discharge or an electromagnetic field so as to stimulate skin along said treatment line.

18. The device as in claim 17, wherein said electrically-powered control unit is configured to produce a sinusoidal voltage waveform having a maximum amplitude ranging from about 7,000 V to 30,000 V at a frequency ranging from about 50 kHz to about 120 kHz, and a maximum output current of less than 0.1 mA.
